(19) United States
(12) Patent Application Publication
(10) Pub. No.: US 2018/0368904 A1
(43) Pub. Date: Dec. 27, 2018

(54) THERMAL ENERGY TREATMENT DEVICE

(71) Applicant: OLYMPUS CORPORATION, Tokyo (JP)

(72) Inventor: Yuta SUGIYAMA, Tokyo (JP)

(73) Assignee: OLYMPUS CORPORATION, Tokyo (JP)

(21) Appl. No.: 16/120,860

(22) Filed: Sep. 4, 2018

Related U.S. Application Data
(63) Continuation of application No. PCT/JP2016/062825, filed on Apr. 22, 2016.

(51) Int. Cl. A61B 18/08

(52) U.S. Cl. CPC A61B 18/082; H05B 1/025

(57) ABSTRACT

A thermal energy treatment device includes: an insulating substrate having a longitudinal axis; a heat generation body that is provided on one surface of the insulating substrate and includes a heater configured to generate heat due to energization and a connector that is electrically connected to the heater; and a processor comprising hardware, the processor being configured to: energize the heater through the connector; estimate a temperature of the connector based on at least one of a current value and a voltage value which are energized to the connector, and a temperature of the heater; and control an output value to be energized to the heater based on the estimated temperature of the connector.

### CROSS-REFERENCE TO RELATED APPLICATION

This application is a continuation of International Application No. PCT/JP2016/062825, filed on Apr. 22, 2016, the entire contents of which are incorporated herein by reference.

### BACKGROUND

### 1. Technical Field

The present disclosure relates to a thermal energy treatment device.

### 2. Related Art

In the related art, there is known a thermal energy treatment device (thermal tissue operation system) that treats (for example, joins (or anastomoses) and separates) a biological tissue by applying energy to the biological tissue (for example, refer to JP 2012-24583 A).

The thermal energy treatment device described in JP 2012-24583 A includes a pair of jaws which grasps the biological tissue. In addition, an energy generation unit that generates thermal energy is provided in the pair of jaws.

For example, with regard to the energy generation unit, it is considered that the energy generation unit is constituted by a flexible substrate and a heat transfer plate to be described below so as to realizing a reduction in thickness.

The flexible substrate is a unit that functions as a sheet heater. In addition, a heater that generates heat due to energization, and a connector that is electrically connected to the heater are formed on one surface of the flexible substrate.

The heat transfer plate is constituted by a conductor such as copper. In addition, the heat transfer plate is provided to face the one surface (heater) of the flexible substrate, and transfers heat generated in the heater to the biological tissue (applies thermal energy to the biological tissue).

Here, the flexible substrate is longer than the heat transfer plate. Accordingly, when being assembled, one end (side in which the connector is provided) of the flexible substrate protrudes from the heat transfer plate. In addition, a lead wire through which electric power is supplied to the heater is connected to the connector that is provided on the one side. That is, the lead wire is placed on the one surface (side in which the heat transfer plate is provided) of the flexible substrate, and thus it is possible to realize a reduction in thickness of the energy generation unit.

### SUMMARY

In some embodiments, a thermal energy treatment device includes: an insulating substrate having a longitudinal axis; a heat generation body that is provided on one surface of the insulating substrate and includes a heater configured to generate heat due to energization and a connector that is electrically connected to the heater, the heater having a first resistance value that is a resistance value per unit length in the longitudinal axis direction, the connector having a second resistance value that is a resistance value per unit length in the longitudinal axis direction and that is smaller than the first resistance value; and a processor comprising hardware, the processor being configured to: energize the heater through the connector; estimate a temperature of the connector based on at least one of a current value and a voltage value which are energized to the connector, and a temperature of the heater; and control an output value to be energized to the heater based on the estimated temperature of the connector.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view schematically illustrating a thermal energy treatment device according to a first embodiment of the disclosure;

FIG. 2 is an enlarged view of a tip end portion (treatment unit) of a treatment tool illustrated in FIG. 1;

FIG. 3 is a view illustrating a first holding member and an energy generation unit which are illustrated in FIG. 2;

FIG. 4 is a view illustrating the first holding member and the energy generation unit which are illustrated in FIG. 2;

FIG. 5 is a block diagram illustrating a control device illustrated in FIG. 1;

FIG. 6 is a flowchart illustrating an operation of the control device illustrated in FIG. 5;

FIG. 7 is a table illustrating a calculation example in Step S8 illustrated in FIG. 6;

FIG. 8 is a graph illustrating an example of first and second weighting coefficients which are used in Steps S7 and S8 illustrated in FIG. 6;

FIG. 9 is a graph illustrating an effect of the first embodiment of the disclosure;

FIG. 10 is a view illustrating a modification example of the first embodiment of the disclosure;

FIG. 11 is a view illustrating a modification example of the first embodiment of the disclosure;

FIG. 12 is a block diagram illustrating a control device that constitutes a thermal energy treatment device according to a second embodiment of the disclosure;

FIG. 13 is a flowchart illustrating an operation of the control device illustrated in FIG. 12;

FIG. 14 is a table illustrating a calculation example in Step S8B illustrated in FIG. 12; and

FIG. 15 is a graph illustrating an effect of the second embodiment of the disclosure.

### DETAILED DESCRIPTION

Hereinafter, an embodiment for carrying out the invention (hereinafter, referred to as “embodiment”) will be described with reference to the accompanying drawings. Furthermore, the invention is not limited to embodiments to be described below. In addition, in description of the drawings, the same reference numeral will be given to the same portions.

### First Embodiment

Schematic Configuration of Thermal Energy Treatment Device

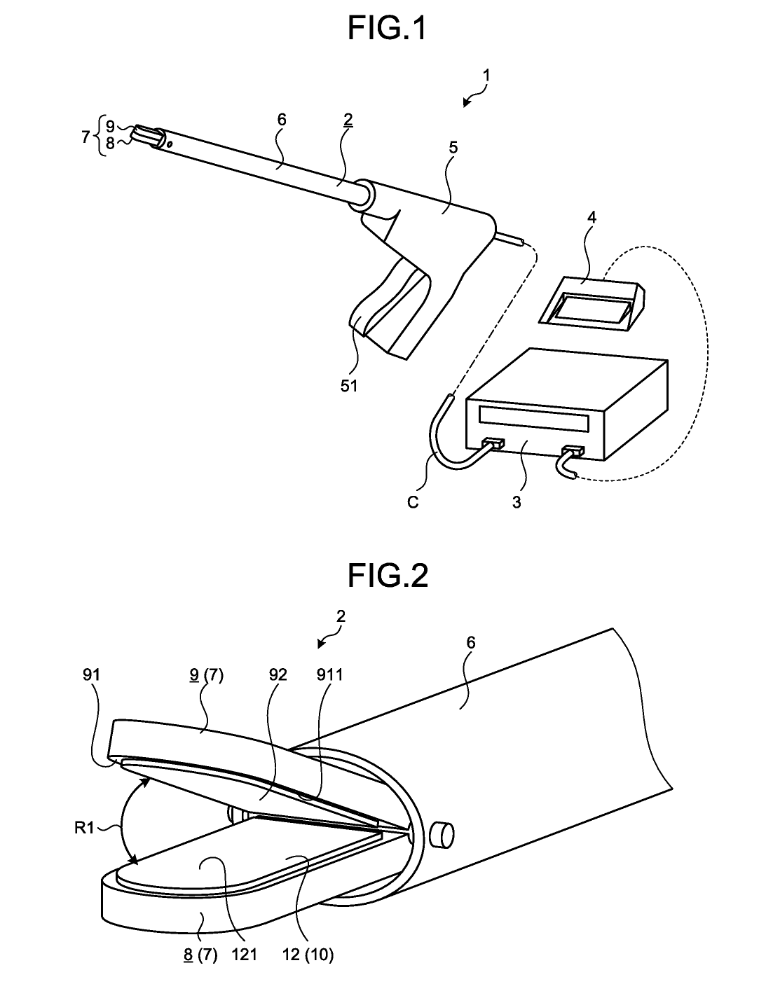

FIG. 1 is a view schematically illustrating a thermal energy treatment device 1 according to a first embodiment of the disclosure.

The thermal energy treatment device 1 applies thermal energy to a biological tissue that is an object to be treated and treats (for example, joins (or anastomoses) and separates) the biological tissue. As illustrated in FIG. 1, the thermal energy treatment device 1 includes a treatment tool 2, a control device 3, and a foot switch 4.

Configuration of Treatment Tool

For example, the treatment tool 2 is a linear type treatment tool for surgery medical treatment that performs treatment with respect to a biological tissue through an abdominal wall. As illustrated in FIG. 1, the treatment tool 2 includes a handle 5, a shaft 6, and a treatment unit 7.

The handle 5 is a portion that is grasped by an operator. In addition, as illustrated in FIG. 1, an operation knob 51 is provided in the handle 5.

As illustrated in FIG. 1, the shaft 6 has an approximately cylindrical shape, and one end (a right end in FIG. 1) is connected to the handle 5. In addition, the treatment unit 7 is formed at the other end (a left end in FIG. 1) of the shaft 6. An opening and closing mechanism (not illustrated), which opens or closes first and second holding members 8 and 9 (FIG. 1) which constitute the treatment unit 7 in correspondence with an operation of the operation knob 51 by an operator, is provided at the inside of the shaft 6. In addition, an electric cable C (FIG. 1) connected to the control device 3 is provided at the inside of the shaft 6 from one end (right end side in FIG. 1) to the other end (left end side in FIG. 1) of the shaft 6 through the handle 5.

Configuration of Treatment Unit

FIG. 2 is an enlarged view of a tip end portion (treatment unit 7) of the treatment tool 2.

The treatment unit 7 is a portion that grasps a biological tissue and treats the biological tissue. As illustrated in FIG. 1 or 2, the treatment unit 7 includes first and second holding members 8 and 9.

The first and second holding members 8 and 9 are pivotally supported to the other end (left end in FIG. 1 and FIG. 2) of the shaft 6 in a manner capable of being opened or closed in a direction indicated by an arrow R1 (FIG. 2), and can grasp a biological tissue in correspondence with an operation of the operation knob 51 by an operator.

Hereinafter, configurations of the first and second holding members 8 and 9 will be sequentially described.

Configuration of First Holding Member

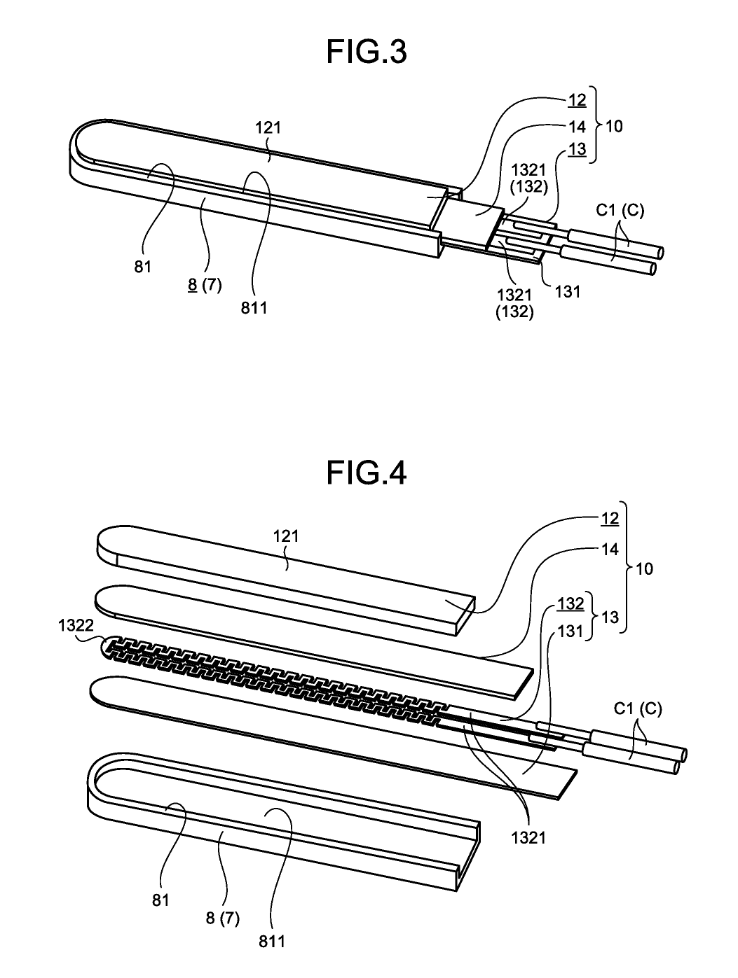

FIG. 3 and FIG. 4 are views illustrating the first holding member 8 and an energy generation unit 10. Specifically, FIG. 3 is a perspective view when the first holding member 8 and the energy generation unit 10 are seen from an upward side in FIG. 1 and FIG. 2. FIG. 4 is an exploded perspective view of FIG. 3.

The first holding member 8 is provided on a downward side in FIG. 1 and FIG. 2 in comparison to the second holding member 9, and has an approximately rectangular shape that extends along a central axis of the shaft 6. Hereinafter, for convenience of description, in the first holding member 8, a surface on an upward side in FIG. 1 or FIG. 4 is described as “first grasping surface 81”.

A first concave portion 811, which is downwardly depressed and extends from one end (right end in FIG. 4) of the first holding member 8 toward the other end thereof along a longitudinal axis direction of the first holding member 8 in FIG. 4, is provided at an approximately central position in a width direction in the first grasping surface 81.

As illustrated in FIG. 2 to FIG. 4, the energy generation unit 10 is provided in the first concave portion 811.

For example, the above-described first holding member 8 is obtained by molding a resin material such as a fluorine resin.

The energy generation unit 10 generates thermal energy under control by the control device 3. As illustrated in FIG. 3 or FIG. 4, the energy generation unit 10 includes a heat transfer plate 12, a flexible substrate 13, and an adhesive sheet 14.

For example, the heat transfer plate 12 is a long thin plate (that extends in the longitudinal axis direction (right and left direction in FIG. 3 and FIG. 4) of the first holding member 8)) that is constituted by a material such as copper. In addition, with regard to the heat transfer plate 12, in a state in which a biological tissue is grasped by the first and second holding members 8 and 9, a treatment surface 121 (a surface on an upward side in FIG. 2 to FIG. 4) that is a surface of the heat transfer plate 12 comes into contact with the biological tissue, and the heat transfer plate 12 transfers heat generated from the flexible substrate 13 to the biological tissue (applies thermal energy to the biological tissue).

A part of the flexible substrate 13 generate heat, and the flexible substrate 13 functions as a sheet heater that heats the heat transfer plate 12 due to the heat generation. As illustrated in FIG. 3 or FIG. 4, the flexible substrate 13 includes an insulating substrate 131 and a wiring pattern 132.

The insulating substrate 131 is a long sheet (extending in the longitudinal axis direction of the first holding member 8) constituted by polyimide that is an insulating material.

Furthermore, as the material of the insulating substrate 131, for example, a high heat-resistant insulating material such as aluminum nitride, alumina, glass, and zirconia may be employed without limitation to polyimide.

Here, a width dimension of the insulating substrate 131 is set to be approximately the same as a width dimension of the heat transfer plate 12. In addition, a longitudinal dimension (a longitudinal dimension in a longitudinal axis direction) of the insulating substrate 131 is set to be longer than a longitudinal dimension (a longitudinal dimension in a longitudinal axis direction) of the heat transfer plate 12.

The wiring pattern 132 is obtained by processing stainless steel (SUS304) that is a conductive material, and includes a pair of connectors 1321 and a heater 1322 (FIG. 4) as illustrated in FIG. 3 or FIG. 4. That is, the wiring pattern 132 has a function as a heat generation body according to the disclosure. In addition, the wiring pattern 132 can be stuck to one surface of the insulating substrate 131 through thermal compression.

Furthermore, the material of the wiring pattern 132 is not limited to stainless steel (SUS304), and may be other stainless steel materials (for example, No. 400 series). In addition, a conductive material such as platinum and tungsten may be employed as the material. In addition, with regard to the wiring pattern 132, there is no limitation to a configuration in which the wiring pattern 132 is stuck to the one surface of the insulating substrate 131 through thermal compression, and a configuration in which the wiring pattern 132 is formed on the one surface through vapor deposition and the like may be employed.

As illustrated in FIG. 3 or FIG. 4, the pair of connectors 1321 extends from one end (right end side in FIG. 4) of the insulating substrate 131 toward the other end thereof, and is provided to face each other in a width direction of the insulating substrate 131. In addition, two lead wires C1, which constitute the electric cable C, are respectively bonded (connected) to the pair of connectors 1321.

One end of the heater 1322 is connected (electrically connected) to one of the connectors 1321, and the heater 1322 extends in a U shape conforming to an outer edge shape of the insulating substrate 131 while meandering from the one end in a wavelike shape. The other end of the heater 1322 is connected (electrically connected) to the other connector 1321.

In addition, when a voltage is applied (energized) to the pair of connectors 1321 through the two lead wires C1 by the control device 3, the heater 1322 generates heat.

Furthermore, according to the first embodiment, in the pair of connectors 1321, an electric resistance value (second resistance value) per unit length in a longitudinal axis direction (right and left direction in FIG. 4) is smaller than an electric resistance value (first resistance value) per unit length of the heater 1322 in a longitudinal axis direction.

As illustrated in FIG. 3 or FIG. 4, the adhesive sheet 14 is provided between the heat transfer plate 12 and the flexible substrate 13, and bonds and fixes a rear surface (surface opposite to the treatment surface 121) of the heat transfer plate 12 and one surface (surface on a wiring pattern 132 side) of the flexible substrate 13 in a state in which a part of the flexible substrate 13 overhangs from the heat transfer plate 12. The adhesive sheet 14 is a long sheet (extending in the longitudinal axis direction of the first holding member 8) that has satisfactory heat conductivity and insulating properties, is durable at a high temperature, and has adhesiveness. For example, the adhesive sheet 14 is formed by mixing a high heat-conductive filler (non-conductive material) such as alumina, boron nitride, graphite, and aluminum nitride with a resin such as epoxy and polyurethane.

Here, a width dimension of the adhesive sheet 14 is set to be approximately the same as the width dimension of the insulating substrate 131. In addition, a longitudinal dimension (longitudinal dimension in a longitudinal axis direction) of the adhesive sheet 14 is set to be longer than the longitudinal dimension (longitudinal dimension in a longitudinal axis direction) of the heat transfer plate 12, and to be shorter than the longitudinal dimension (longitudinal dimension in a longitudinal axis direction) of the insulating substrate 131.

In addition, the heat transfer plate 12 is disposed to cover the entire region of the heater 1322. In addition, the adhesive sheet 14 is disposed to cover the entire region of the heater 1322 and to cover a part of the pair of connectors 1321. That is, the adhesive sheet 14 is disposed in a state of overhanging to the right relatively to the heat transfer plate 12 in FIG. 3 and FIG. 4. In addition, the two lead wires C1 (FIG. 3 and FIG. 4) are respectively bonded (connected) to portions (portions not covered with the adhesive sheet 14) exposed to the outside in the pair of connectors 1321.

Configuration of Second Holding Member

As illustrated in FIG. 2, the second holding member 9 has approximately the same external shape as that of the first holding member 8. Hereinafter, with regard to the second holding member 9, a surface (surface on a downward side in FIG. 2) that faces the first grasping surface 81 is described as “second grasping surface 91”.

As in the first holding member 8, a second concave portion 911, which is upwardly depressed and extends from the one end (right end in FIG. 2) of the second holding member 9 toward the other end thereof along a longitudinal axis direction of the second holding member 9 in FIG. 2, is provided at an approximately central position in a width direction in the second grasping surface 91.

As illustrated in FIG. 2, a heat transfer plate 92 that is the same as the heat transfer plate 12 is provided in the second concave portion 911.

Configuration of Control Device and Foot Switch

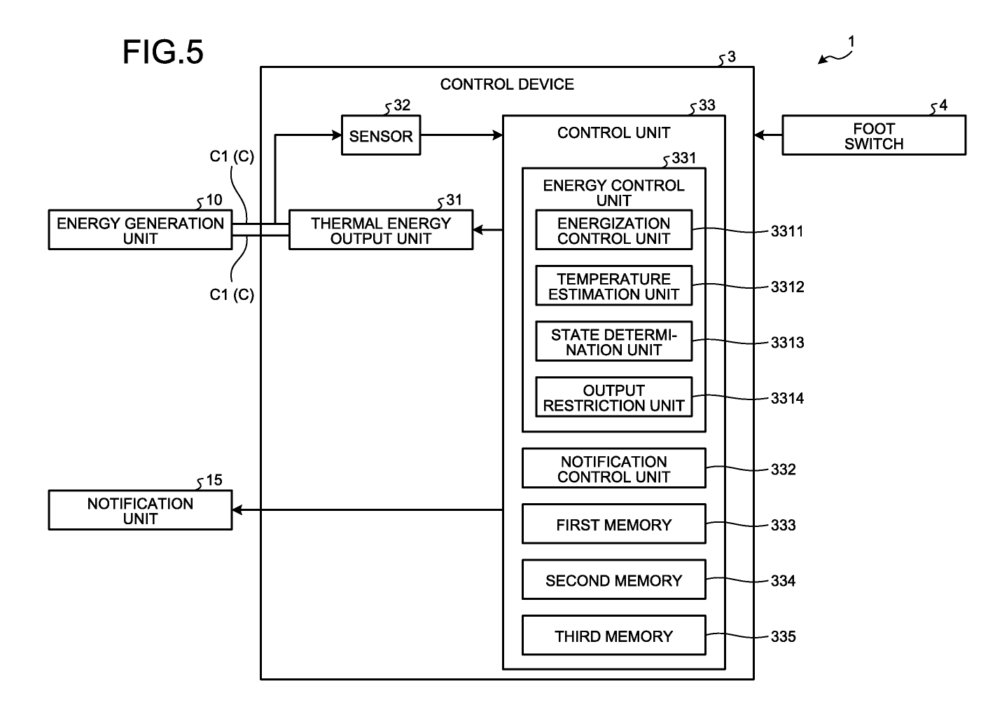

FIG. 5 is a block diagram illustrating the control device 3.

Furthermore, in FIG. 5, as a configuration of the thermal energy treatment device 1 (control device 3), a main portion of the disclosure is mainly illustrated in the drawing.

When being pressed (turned ON) by an operator's foot, the foot switch 4 receives a first user operation of transitioning the treatment tool 2 from a standby state (state of waiting for treatment for a biological tissue) to a treatment state (state of treating the biological tissue). In addition, when the operator's foot is detached from the foot switch 4 (turned OFF), the foot switch 4 receives a second user operation of transitioning the treatment tool 2 from the treatment state to the standby state. In addition, the foot switch 4 outputs signals corresponding to the first and second user operations to the control device 3.

Furthermore, as a configuration that receives the first and second user operations, a switch that is operated by a hand, and the like may be employed without limitation to the foot switch 4.

The control device 3 collectively controls an operation of the treatment tool 2. As illustrated in FIG. 5, the control device 3 includes a thermal energy output unit 31, a sensor 32, and a control unit 33.

The thermal energy output unit 31 applies (energizes) a voltage to the energy generation unit 10 (wiring pattern 132) through the two lead wires C1 under control by the control unit 33.

The sensor 32 detects a current value and a voltage value which are supplied (energized) from the thermal energy output unit 31 to the energy generation unit 10. In addition, the sensor 32 outputs signals corresponding to the current value and the voltage value, which are detected, to the control unit 33.

The control unit 33 includes a central processing unit (CPU) and the like, and executes a feedback control of the energy generation unit 10 (wiring pattern 132) in accordance with a predetermined control program. As illustrated in FIG. 5, the control unit 33 includes an energy control unit 331, a notification control unit 332, and first to third memories 333 to 335.

The energy control unit 331 controls an output value (electric power value) to be supplied (energized) to the energy generation unit 10. As illustrated in FIG. 5, the energy control unit 331 includes an energization control unit 3311, a temperature estimation unit 3312, a state determination unit 3313, and an output restriction unit 3314.

In a case where the foot switch 4 is turned ON (in a case where the foot switch 4 receives the first user operation), the energization control unit 3311 switches the treatment tool 2 to the treatment state.

Specifically, in a case where the treatment tool 2 is switched to the treatment state, the energization control unit 3311 supplies an output value (electric power value) necessary to set the energy generation unit 10 to a target temperature to the energy generation unit 10 (wiring pattern 132) through the thermal energy output unit 31 (performs a feedback control of the energy generation unit 10) while grasping a temperature (hereinafter, described as “heater temperature”) of the heater 1322.

Here, the heater temperature that is used in the feedback control is a temperature that is calculated as described below.

Specifically, a resistance value of the wiring pattern 132 is acquired on the basis of a current value and a voltage value which are detected by the sensor 32 (a current value and a voltage value which are supplied (energized) from the thermal energy output unit 31 to the energy generation unit 10 (wiring pattern 132)). In addition, the resistance value of the wiring pattern 132 is converted into a temperature by using a relationship between the resistance value and the temperature of the wiring pattern 132 which are calculated in advance through an experiment. In addition, the temperature is referred to as “heater temperature”.

In addition, in a case where the foot switch 4 is turned OFF (in a case where the foot switch 4 receives the second user operation), the energization control unit 3311 switches the treatment tool 2 to the standby state.

Specifically, in a case where the treatment tool 2 is switched to the standby state, the energization control unit 3311 supplies a minimum output electric power (for example, 0.1 W) to the energy generation unit 10 (wiring pattern 132) through the thermal energy output unit 31 to acquire the heater temperature (to detect the current value and the voltage value by the sensor 32).

The temperature estimation unit 3312 estimates a temperature of the connectors 1321 (a temperature of a portion not covered with the adhesive sheet 14 (exposed from the adhesive sheet 14) in the connectors 1321) on the basis of information stored in the first to third memories 333 to 335. Furthermore, the estimated temperature of the connectors 1321 is described as “estimation temperature”.

Here, the first memory 333 sequentially stores the heater temperature calculated for every predetermined sampling interval (for example, 0.05 seconds (hereinafter, a second is described as “s”) by the energy control unit 331 (energization control unit 3311) on the basis of the current value and the voltage value which are detected by the sensor 32 in association with time at which the heater temperature is calculated. That is, the first memory 333 has a function as a first storage unit according to the disclosure.

Furthermore, the first memory 333 sequentially stores only heater temperatures which are calculated from the current time to a past before a predetermined time (time equal to an integral time to be described below). That is, in a case where the heater temperature is newly calculated, and the latest heater temperature is stored in the first memory 333, the oldest heater temperature is erased.

In addition, the second memory 334 sequentially stores the current value that is detected by the sensor 32 in association with time at which the current value is detected. That is, the second memory 334 has a function as a third storage unit according to the disclosure.

Furthermore, as in the first memory 333, the second memory 334 sequentially stores only current values which are detected from the current time to a past before a predetermined time (time equal to an integral time to be described below). That is, in a case where the current value is newly detected, and the latest current value is stored in the second memory 334, the oldest current value is erased.

In addition, the third memory 335 is constituted by a nonvolatile memory, and stores a control program that is executed by the control unit 33, and an assumed environment temperature (approximately 37° C. to 40° C. because use in a living body is assumed) on an outer side of the treatment tool 2. In addition, the third memory 335 stores a plurality of first and second weighting coefficients calculated in advance through an experiment in association with time going back to the past from the current time. That is, the third memory 335 has a function as second and fourth storage units according to the disclosure.

The state determination unit 3313 determines a state of the connector 1321 on the basis of a temperature estimated by the temperature estimation unit 3312.

Specifically, the state determination unit 3313 compares the temperature estimated by the temperature estimation unit 3312 and a temperature restriction value (temperature at which it is determined that the connector 1321 enters an overheating state) that is set in advance, and determines whether or not the estimation temperature is equal to or higher than the temperature restriction value. In addition, in a case where it is determined that the estimation temperature is equal to or higher than the temperature restriction value, the state determination unit 3313 sets a timer (initial value is 0) to a defined time (for example, 3 s). In addition, in a case where it is determined that the estimation temperature is lower than the temperature restriction value, the state determination unit 3313 counts down the timer, and determines whether or not a value of the timer is 0 or less.

The output restriction unit 3314 restricts (controls) an output value (electric power value) to be supplied (energized) to the energy generation unit 10 (wiring pattern 132) on the basis of a determination result of the state determination unit 3313. That is, the output restriction unit 3314 has a function as an output control unit according to the disclosure.

The notification control unit 332 controls an operation of a notification unit 15 (FIG. 5) on the basis of a determination result of the state determination unit 3313.

In the first embodiment, the notification unit 15 is constituted by a speaker that gives a notification of predetermined information (generates an alarm sound) by a voice. Furthermore, for example, the notification unit 15 may be constituted by a display that displays predetermined information, a light emitting diode (LED) that gives a notification of predetermined information through lighting or flickering, and the like without limitation to the speaker.

Operation of Control Device

Next, description will be given of an operation of the above-described control device 3.

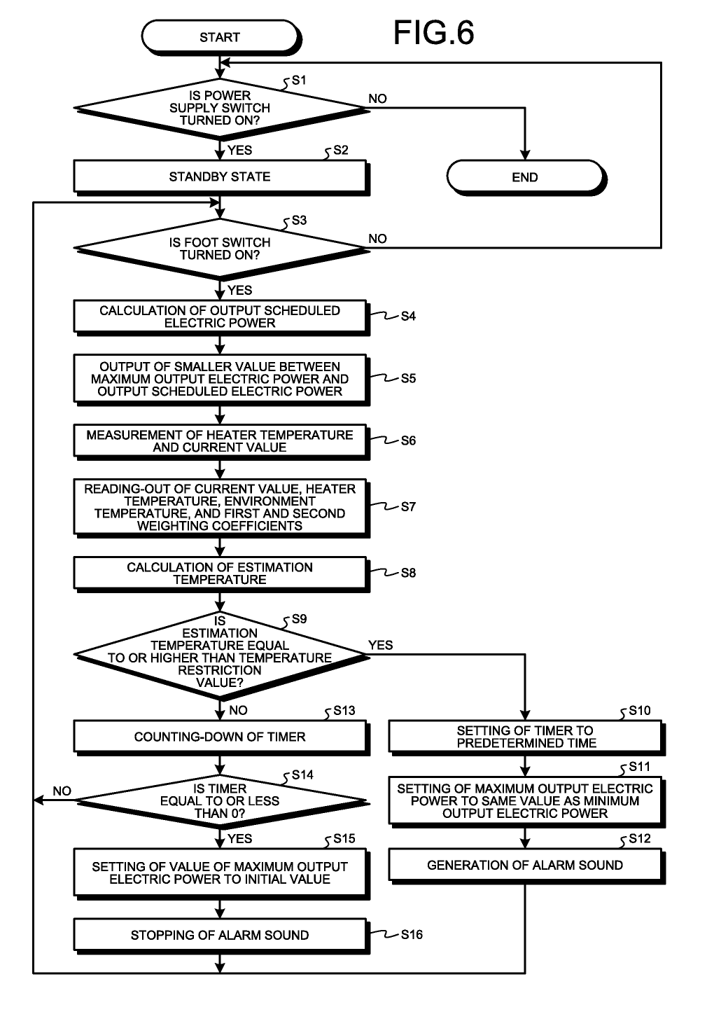

FIG. 6 is a flowchart illustrating an operation of the control device 3.

After a power supply switch (not illustrated) of the thermal energy treatment device 1 (control device 3) is turned on by an operator (Step S1: Yes), the energization control unit 3311 switches the treatment tool 2 to the standby state (Step S2).

Specifically, in Step S2, the energization control unit 3311 supplies (energizes) minimum output electric power (for example, 0.1 W) to the energy generation unit 10 through the thermal energy output unit 31. That is, in this state, it enters a state in which the heater temperature can be acquired (the current value and the voltage value can be detected by the sensor 32).

After Step S2, the control unit 33 determines whether or not the foot switch 4 is turned ON (Step S3).

In a case where it is determined that the foot switch 4 is turned OFF (or an OFF state continues) (Step S3: No), the control device 3 returns to Step S1.

On the other hand, in a case where it is determined that the foot switch 4 is turned ON (Step S3: Yes), the energization control unit 3311 switches the treatment tool 2 to the treatment state (Steps S4 and S5).

Specifically, in Step S4, the energization control unit 3311 calculates an output value (output scheduled electric power) that is necessary to set the energy generation unit 10 to a target temperature while grasping the heater temperature. In addition, in Step S5, the energization control unit 3311 supplies (energizes) a smaller value between the output scheduled electric power and the maximum output electric power (for example, an initial value is 100 W) to the energy generation unit 10 through the thermal energy output unit 31.

After Step S5, the energization control unit 3311 measures a current heater temperature, and a current value that is supplied (energized) to the energy generation unit 10 (Step S6).

Specifically, in Step S6, the energization control unit 3311 calculates the heater temperature on the basis of a current value and a voltage value which are detected by the sensor 32. In addition, the energization control unit 3311 stores the calculated heater temperature in the first memory 333 in association with time at which the heater temperature is calculated. In addition, the energization control unit 3311 stores the current value that is detected by the sensor 32 in the second memory 334 in association with time at which the current value is detected.

After Step S6, the temperature estimation unit 3312 reads out the current value, the heater temperature, the environment temperature, and the first and second weighting coefficients from the first to third memories 333 to 335 (Step S7).

After Step S7, the temperature estimation unit 3312 substitutes the current value, the heater temperature, the environment temperature, and the first and second weighting coefficients, which are read out, for the following Formula (1) to calculate an estimation temperature (Step S8).

$${T}_{conduction} = {\sum }_{t = 0}^{{Period}_{—} max} \{ α ( t ) {I ( t )}^{2} Δ t + β ( t ) ( {T}_{heater} ( t ) - {T}_{atmosphere} ) Δ t \} + {T}_{atmosphere} \qquad ( 1 )$$

Here, in Mathematical Formula (1), T_{conduction} represents an estimation temperature [° C.] to be calculated. Period_max represents an integral time [s]. “t” represents time going back to the past from the current time (time t at the current time is 0 s, and time t previous to (before) the current time is a negative value). α(t) represents a second weighting coefficient [A²/(° C.·s)] relating to the time t going back to the past from the current time. I(t) represents a current value (current value [A] detected by the sensor 32) relating to the time t going back to the past from the current time. β(t) represents a first weighting coefficient [1/s] relating to the time t going back to the past from the current time. Tₕₑₐₜₑᵣ (t) represents a heater temperature relating to the time t going back to the past from the current time. Tₐₜₘₒₛₚₕₑᵣₑ represents an assumed environment temperature at the outside of the treatment tool 2. Δt represents a sampling interval (for example, 0.05 s).

Hereinafter, description will be given of a calculation example of the estimation temperature T_{conduction} in Step S8.

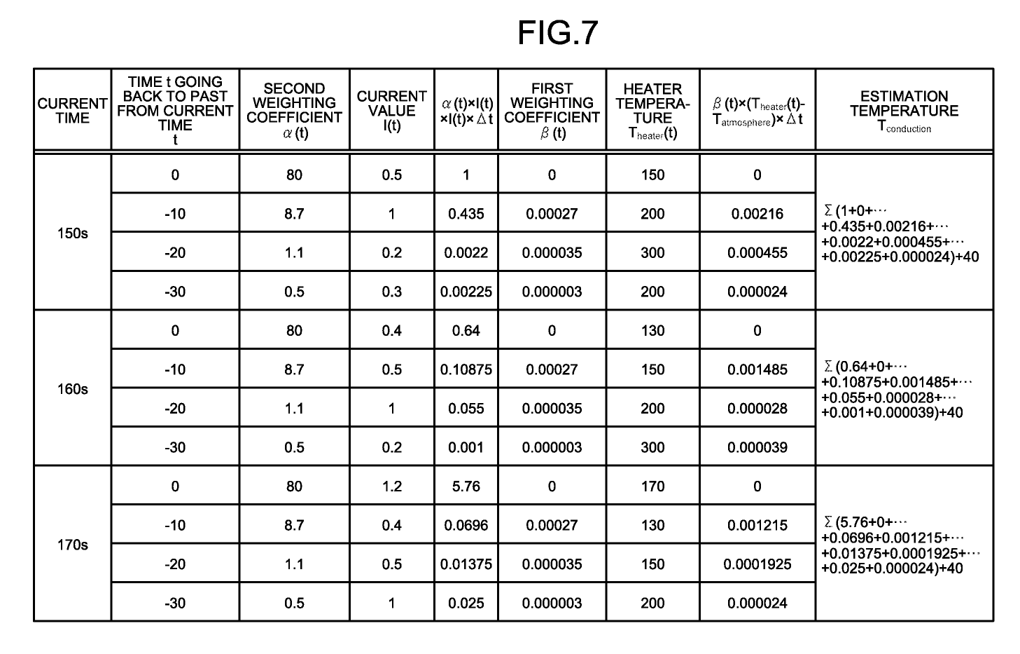

FIG. 7 is a table illustrating the calculation example in Step S8. Furthermore, in calculation of Formula (1), a value for every sampling interval Δt is used, but in FIG. 7, only values at t=0 s, −10 s, −20 s, and −30 s are described for convenience of explanation.

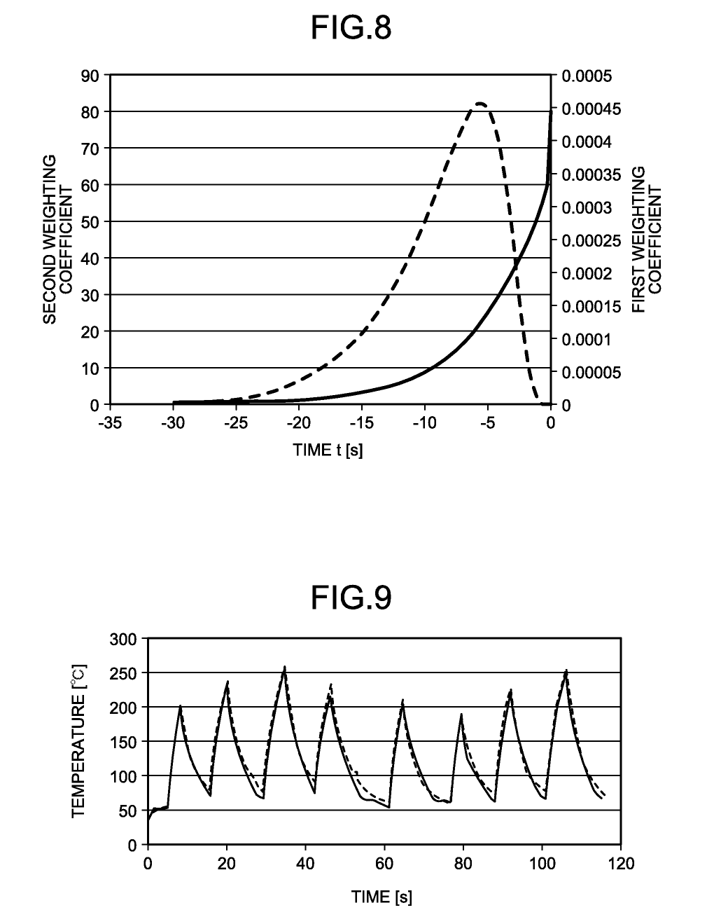

In the example in FIG. 7, the integral time Period_max is set to 30 s, the environment temperature Tₐₜₘₒₛₚₕₑᵣₑ is set to 40° C., and the sampling interval Δt is set to 0.05 s. In addition, the second weighting coefficients α [A²/(° C.·s)] at t=0 s, −10 s, −20 s, and −30 s are respectively set to “80”, “8.7”, “1.1”, and “0.5” (refer to a solid line in FIG. 8). In addition, the first weighting coefficients β [l/s] at t=0 s, −10 s, −20 s, and −30 s are respectively set to “0”, “0.00027”, “0.000035”, and “0.000003” (refer to a broken line in FIG. 8).

In addition, in the example in FIG. 7, when the current time is 150 s (for example, time after the foot switch 4 is turned ON), current values I, which are detected by the sensor 32 at the current time (t=0 s), and times (t=−10 s, −20 s, and −30 s) before 10 s, 20 s, and 30 s from the current time, are respectively set to 0.5 A, 1 A, 0.2 A, and 0.3 A.

In addition, in the example in FIG. 7, when the current time is 150 s, the heater temperatures Tₕₑₐₜₑᵣ, which are respectively calculated at t=0 s, −10 s, −20 s, and −30 s by the energization control unit 3311, are set to 150° C., 200° C., 300° C., and 200° C.

In addition, when the current time is 150 s, in Step S8, the estimation temperature T_{conduction} is calculated as follows by using Formula (1).

That is, the temperature estimation unit 3312 calculates each difference between the environment temperature Tₐₜₘₒₛₚₕₑᵣₑ and each heater temperature Tₕₑₐₜₑᵣ (t) for every sampling interval Δt at the integral time Period_max on the basis of Formula (1), multiplies the difference by the first weighting coefficient β(t) at corresponding times t, and integrate a resultant value of the multiplying to obtain a first integrated value. In addition, the temperature estimation unit 3312 multiplies the square of each current value I(t) for every sampling interval Δt at the integral time Period_max by the second weighting coefficient α(t) at corresponding times t and integrate a resultant value of the multiplying to obtain a second integrated value. In addition, the first integrated value, the second integrated value, the environment temperature Tₐₜₘₒₛₚₕₑᵣₑ are added together to calculate the estimation temperature T_{conduction}.

Specifically, α(t)×I(t)×I(t)×Δt at t=0 s becomes 80×0.5×0.5×0.05=1. In addition, α(t)×I(t)×I(t)×Δt at t=−10 s becomes 8.7×1×1×0.05=0.435. In addition, α(t)×I(t)×I(t)×Δt at t=−20 s becomes 1.1×0.2×0.2×0.05=0.0022. In addition, α(t)×I(t)×I(t)×Δt at t=−30 s becomes 0.5×0.3×0.3×0.05=0.00225.

On the other hand, β(t)×(Tₕₑₐₜₑᵣ (t)−Tₐₜₘₒₛₚₕₑᵣₑ)×Δt at t=0 s becomes 0×(150−40)×0.05=0. In addition, β(t)×(Tₕₑₐₜₑᵣ (t)−Tₐₜₘₒₛₚₕₑᵣₑ)×Δt at t=−10 s becomes 0.00027×(200−40)×0.05=0.00216. In addition, β(t)×(Tₕₑₐₜₑᵣ (t)−Tₐₜₘₒₛₚₕₑᵣₑ)×Δt at t=−20 s becomes 0.00035×(300−40)×0.05=0.000455. In addition, β(t)×(Tₕₑₐₜₑᵣ (t)−Tₐₜₘₒₛₚₕₑᵣₑ)×Δt at t=−30 s becomes 0.00003×(200−40)×0.05=0.000024.

In addition, respective values of α(t)×I(t)×I(t)×Δt and respective values of β(t)×(Tₕₑₐₜₑᵣ (t)−Tₐₜₘₒₛₚₕₑᵣₑ)×Δt at t=0 s, −0.05 s, −0.1 s, −0.15 s, . . . −30 s at the integral time Period_max are added together, and the environment temperature Tₐₜₘₒₛₚₕₑᵣₑ (=40° C.) is added to the resultant added value to calculate the estimation temperature T_{conduction} at the current time of 150 s.

In addition, in the example in FIG. 7, a current value I detected by the sensor 32 when the current time is 160 s (t=0 s) is set to 0.4 A. Furthermore, in a case where the current time is 160 s, as 10 seconds has passed since the current time was 150 s, current values I which are respectively detected by the sensor 32 before the current time (160 s) by 10 s, 20 s, and 30 s (t=−10 s, −20 s, and −30 s) are the same values as the current values I at t=0 s, −10 s, and −20 s in a case where the current time is 150 s.

In addition, in the example in FIG. 7, the heater temperature Tₕₑₐₜₑᵣ calculated by the energization control unit 3311 when the current time is 160 s (t=0 s) is set to 130° C. Furthermore, in a case where the current time is 160 s, heater temperatures Tₕₑₐₜₑᵣ calculated by the energization control unit 3311 at t=−10 s, −20 s, and −30 s are the same values as the heater temperatures Tₕₑₐₜₑᵣ at t=0 s, −10 s, and −20 s in a case where the current time is 150 s.

In addition, when the current time is 160 s, in Step S8, the estimation temperature T_{conduction} is calculated as follows by using Formula (1).

Specifically, α(t)×I(t)×I(t)×Δt at t=0 s becomes 80×0.4×0.4×0.05=0.64. In addition, α(t)×I(t)×I(t)×Δt at t=−10 s becomes 8.7×0.5×0.5×0.05=0.10875. In addition, α(t)×I(t)×I(t)×Δt at t=−20 s becomes 1.1×1×1×0.05=0.055. In addition, ×(t)×I(t)×I(t)×Δt at t=−30 s becomes 0.5×0.2×0.2×0.05=0.001.

On the other hand, β(t)×(Tₕₑₐₜₑᵣ (t)−Tₐₜₘₒₛₚₕₑᵣₑ)×Δt at t=0 s becomes 0×(130−40)×0.05=0. In addition, β(t)×(Tₕₑₐₜₑᵣ (t)−Tₐₜₘₒₛₚₕₑᵣₑ)×Δt at t=−10 s becomes 0.00027×(150−40)×0.05=0.001485. In addition, β(t)×(Tₕₑₐₜₑᵣ (t)−Tₐₜₘₒₛₚₕₑᵣₑ)×Δt at t=−20 s becomes 0.00035×(200−40)×0.05=0.000028. In addition, β(t)×(Tₕₑₐₜₑᵣ (t)−Tₐₜₘₒₛₚₕₑᵣₑ)×Δt at t=−30 s becomes 0.00003×(300−40)×0.05=0.000039.

In addition, respective values of α(t)×I(t)×I(t)×Δt and respective values of β(t)×(Tₕₑₐₜₑᵣ (t)−Tₐₜₘₒₛₚₕₑᵣₑ)×Δt at t=0 s, −0.05 s, −0.1 s, −0.15 s, . . . −30 s at the integral time Period_max are added together, and the environment temperature Tₐₜₘₒₛₚₕₑᵣₑ (=40° C.) is added to the resultant added value to calculate the estimation temperature T_{conduction} at the current time of 160 s.

In addition, in the example in FIG. 7, a current value I detected by the sensor 32 when the current time is 170 s (t=0 s) is set to 1.2 A. Furthermore, in a case where the current time is 170 s, as 10 seconds has passed since the current time was 160 s, current values I which are respectively detected by the sensor 32 before the current time (170 s) by 10 s, 20 s, and 30 s (t=−10 s, −20 s, and −30 s) are the same values as the current values I at t=0 s, −10 s, and −20 s in a case where the current time is 160 s.

In addition, in the example in FIG. 7, the heater temperature Tₕₑₐₜₑᵣ calculated by the energization control unit 3311 when the current time is 170 s (t=0 s) is set to 170° C. Furthermore, in a case where the current time is 170 s, heater temperatures Tₕₑₐₜₑᵣ calculated by the energization control unit 3311 at t=−10 s, −20 s, and −30 s are the same values as the heater temperatures Tₕₑₐₜₑᵣ at t=0 s, −10 s, and −20 s in a case where the current time is 160 s.

In addition, when the current time is 170 s, in Step S8, the estimation temperature T_{conduction} is calculated as follows by using Formula (1).

Specifically, α(t)×I(t)×I(t)×Δt at t=0 s becomes 80×1.2×1.2×0.05=5.76. In addition, α(t)×I(t)×I(t)×Δt at t=−10 s becomes 8.7×0.4×0.4×0.05=0.0696. In addition, α(t)×I(t)×I(t)×Δt at t=−20 s becomes 1.1×0.5×0.5×0.05=0.01375. In addition, α(t)×I(t)×I(t)×Δt at t=−30 s becomes 0.5×1×1×0.05=0.025.

On the other hand, β(t)×(Tₕₑₐₜₑᵣ (t)−Tₐₜₘₒₛₚₕₑᵣₑ)×Δt at t=0 s becomes 0×(170−40)×0.05=0. In addition, (t)×(Tₕₑₐₜₑᵣ (t)−Tₐₜₘₒₛₚₕₑᵣₑ)×Δt at t=−10 s becomes 0.00027×(130−40)×0.05=0.001215. In addition, β(t)×(Tₕₑₐₜₑᵣ (t)−Tₐₜₘₒₛₚₕₑᵣₑ)×Δt at t=−20 s becomes 0.00035×(150−40)×0.05=0.0001925. In addition, β(t)×(Tₕₑₐₜₑᵣ (t)−Tₐₜₘₒₛₚₕₑᵣₑ)×Δt at t=−30 s becomes 0.00003×(200−40)×0.05=0.000024.

In addition, respective values of α(t)×I(t)×I(t)×Δt and respective values of β(t)×(Tₕₑₐₜₑᵣ (t)−Tₐₜₘₒₛₚₕₑᵣₑ)×Δt at t=0 s, −0.05 s, −0.1 s, −0.15 s, . . . −30 s at the integral time Period_max are added together, and the environment temperature Tₐₜₘₒₛₚₕₑᵣₑ (=40° C.) is added to the resultant added value to calculate the estimation temperature T_{conduction} at the current time of 170 s.

Hereinafter, description will be given of a calculation method of the first weighting coefficient β(t) and the second weighting coefficient α(t) which are used in Steps S7 and S8.

FIG. 8 is a graph illustrating an example of the first weighting coefficient β(t) and the second weighting coefficient α(t) which are used in Steps S7 and S8. Specifically, in FIG. 8, the horizontal axis represents a time t (a time t at the current time is 0 s, and a time t previous to (before) the current time is a negative value) going back to the past from the current time, and the vertical axis represents the first and second weighting coefficients. Furthermore, in FIG. 8, the first weighting coefficient β(t) is indicated by a broken line, and the second weighting coefficient α(t) is indicated by a solid line. In addition, the first weighting coefficient β(t) and the second weighting coefficient α(t) which are illustrated in FIG. 8 are the first weighting coefficient β(t) and the second weighting coefficient α(t) which are respectively used in the calculation example of the estimation temperature T_{conduction} in FIG. 7.

As described above, the first weighting coefficient β(t) and the second weighting coefficient α(t) which are used in Steps S7 and S8 are values which are calculated in advance through an experiment and are stored in the third memory 335.

In the experiment, first, the first weighting coefficient β(t) is calculated.

Specifically, the heater temperature Tₕₑₐₜₑᵣ is measured from a resistance value of the wiring pattern 132 on the basis of a current value and a voltage value which are detected by the sensor 32 for every sampling interval Δt while energizing a minute current (for example, 1 mA) to the wiring pattern 132, and heating the heater 1322 by using another heat generation source on an outer side, and a temperature (T_{conduction}) of the connectors 1321 is measured (actually measured) by a temperature sensor (not illustrated). In addition, the heater temperature Tₕₑₐₜₑᵣ and the temperature (T_{conduction}) of the connectors 1321 which are measured and the environment temperature are substituted for Formula (1) and are inverted to calculate the first weighting coefficient β(t). Furthermore, in the inversion, the current value I that is energized to the wiring pattern 132 is minute (for example, 1 mA), and thus the term of α(t)×I(t)×I(t)×Δt in Formula (1) is regarded as “0”.

As indicated by the broken line in FIG. 8, as a tendency of the first weighting coefficient β(t), a peak exists immediately before t=0 s at the current time (in the example in FIG. 8, t is approximately −6 s), and the first weighting coefficient β(t) approaches to 0 from the peak as the time t proceeds to the past.

Furthermore, in Formula (1), the term of β(t)×(Tₕₑₐₜₑᵣ (t)−Tₐₜₘₒₛₚₕₑᵣₑ)×Δt is a term that has an effect on the temperature of the connectors 1321 due to heat exchange with the outside of the treatment tool 2 or the heater 1322. According to this, as in the tendency that is also shown in the first weighting coefficient β(t) (a peak exists immediately before the current time t=0 s), the term is influenced by a temperature of the heater 1322 and the like after elapse of a predetermined time.

Next, the second weighting coefficient α(t) is calculated.

Specifically, as described above, after calculating the first weighting coefficient β(t), the heater temperature Tₕₑₐₜₑᵣ is measured from the resistance value of the wiring pattern 132 on the basis of the current value and the voltage value which are detected by the sensor 32 for every sampling interval Δt while changing the current value I, and the temperature (T_{conduction}) of the connectors 1321 is measured (actually measured) by a temperature sensor (not illustrated). In addition, the heater temperature Tₕₑₐₜₑᵣ and the temperature (T_{conduction}) of the connectors 1321 which are measured, the environment temperature, and the first weighting coefficient β(t) calculated as described above are substituted for Formula (1) and are inverted to calculate the second weighting coefficient α(t).

As indicated by the solid line in FIG. 8, as a tendency of the second weighting coefficient α(t), the highest value exists at t=0 s at the current time, and the second weighting coefficient α(t) approaches to 0 from the highest value as the time t proceeds to the past.

Furthermore, the term of α(t)×I(t)×I(t)×Δt in Formula (1) is a term that has an effect on the temperature of the connectors 1321 due to heat generation of the connectors 1321 in correspondence with energization to the wiring pattern 132 (the connectors 1321 and the heater 1322). According to this, as in the tendency that is also shown in the second weighting coefficient α(t) (the highest value exists at the current time t=0 s), the term is immediately influenced by the energization after the energization.

In the first embodiment, a time t, at which the first weighting coefficient β(t) and the second weighting coefficient α(t) become values which are approximately 1/100 times the maximum values thereof, is set as the integral time Period_max. Specifically, at a time of t=−30 s, the first weighting coefficient β(t) and the second weighting coefficient α(t) are approximately “0”. According to this, in the calculation example of the estimation temperature T_{conduction} in FIG. 7, the integral time Period_max is set to 30 s.

Returning to FIG. 6, description of the operation of the control device 3 continues.

After Step S8, the state determination unit 3313 determines whether or not the estimation temperature T_{conduction} that is calculated in Step S8 is equal to or higher than the temperature restriction value (Step S9).

In a case where it is determined that the estimation temperature T_{conduction} that is calculated in Step S8 is equal to or higher than the temperature restriction value (Step S9: Yes), the state determination unit 3313 sets a timer to a predetermined time (for example, 3 s) (Step S10).

After Step S10, the output restriction unit 3314 sets the maximum output electric power (for example, an initial value is 100 W) to the same value as the minimum output electric power (for example, 0.1 W) (Step S11).

As described above, in a case where the treatment tool 2 is switched to the treatment state, in Step S5, a smaller value between the output scheduled electric power and the maximum output electric power is supplied (energized) to the energy generation unit 10. According to this, the output restriction unit 3314 sets the maximum output electric power (for example, an initial value is 100 W) to the minimum output electric power (for example, 0.1 W) in Step S11 to restrict (output restriction) an output value (electric power value) to be supplied (energized) to the energy generation unit 10 to the minimum output electric power (for example, 0.1 W).

After Step S11, the notification control unit 332 generates an alarm sound by operating the notification unit 15 (Step S12). Next, the control device 3 returns to Step S3.

On the other hand, in a case where it is determined that the estimation temperature T_{conduction} that is calculated in Step S8 is lower than the temperature restriction value (Step S9: No), the state determination unit 3313 counts down the timer (Step S13).

Specifically, in a case where the timer is 0 that is an initial value, the state determination unit 3313 sets the timer to a negative value by performing counting-down in Step S13. In addition, after the timer is set to a predetermined time (for example, 3 s), the state determination unit 3313 counts down the timer from the predetermined time in Step S13.

After Step S13, the state determination unit 3313 determines whether or not the timer is 0 or less (Step S14).

In a case where it is determined that the timer is not 0 or less (Step S14: No), the control device 3 returns to Step S3.

On the other hand, in a case where it is determined that the timer is 0 or less (Step S14: Yes), the output restriction unit 3314 sets the maximum output electric power to an initial value (for example, 100 W) (Step S15).

That is, in a case where output restriction is performed in Step S11, the output restriction unit 3314 releases the output restriction in Step S15. In addition, in a case where the output restriction is not performed in Step S11, the maximum output electric power is maintained to the initial value in Step S15.

After Step S15, the notification control unit 332 stops the operation of the notification unit 15, and stops the alarm sound (Step S16). Next, the control device 3 returns to Step S3.

That is, in a case where the alarm sound is generated in Step S12, the notification control unit 332 stops the alarm sound in Step S16. In addition, in a case where alarm sound is not generated in Step S12, an alarm-sound stopping state continues in Step S16.

In the thermal energy treatment device 1 according to the first embodiment as described above, the estimation temperature T_{conduction} is calculated on the basis of the current value I that is energized to the energy generation unit 10, and the heater temperature Tₕₑₐₜₑᵣ, an output value that is energized to the energy generation unit 10 is restricted on the basis of the estimation temperature T_{conduction}.

Accordingly, it is possible to determine whether or not the pair of connectors 1321 can enter an overheating state. In addition, in a case where it is determined that the pair of connectors 1321 can enter the overheating state, it is possible to avoid the overheating state of the pair of connectors 1321 by restricting the output value that is energized to the energy generation unit 10.

In addition, a configuration in which a temperature sensor is provided and a temperature of the connectors 1321 is actually measured by the temperature sensor can be considered. However, in the first embodiment, it is possible to calculate the estimation temperature T_{conduction} without providing the temperature sensor, and thus it is possible to simplify the structure of the treatment tool 2.

FIG. 9 is a graph illustrating an effect of the first embodiment of the disclosure. Specifically, FIG. 9 is a graph obtained by plotting the estimation temperature T_{conduction} (indicated by a solid line in FIG. 9) that is calculated by the temperature estimation unit 3312 and the temperature (indicated by a broken line in FIG. 9) of the connectors 1321 which is measured (actually measured) by a temperature sensor (not illustrated) while increasing or decreasing the current value I that is energized to the energy generation unit 10.

Particularly, in the thermal energy treatment device 1 according to the first embodiment, the estimation temperature T_{conduction} is calculated by Formula (1) in which consideration is given to heat exchange with the outside of the treatment tool 2 or between the heater 1322 and the connectors 1321, and heat generation of the connectors 1321 in correspondence with the energization.

According to this, as illustrated in FIG. 9, it is possible to calculate the estimation temperature T_{conduction} (solid line in FIG. 9) with high accuracy so that the estimation temperature T_{conduction} becomes approximately the same temperature as the actually measured temperature (broken line in FIG. 9).

### Modification Example of First Embodiment

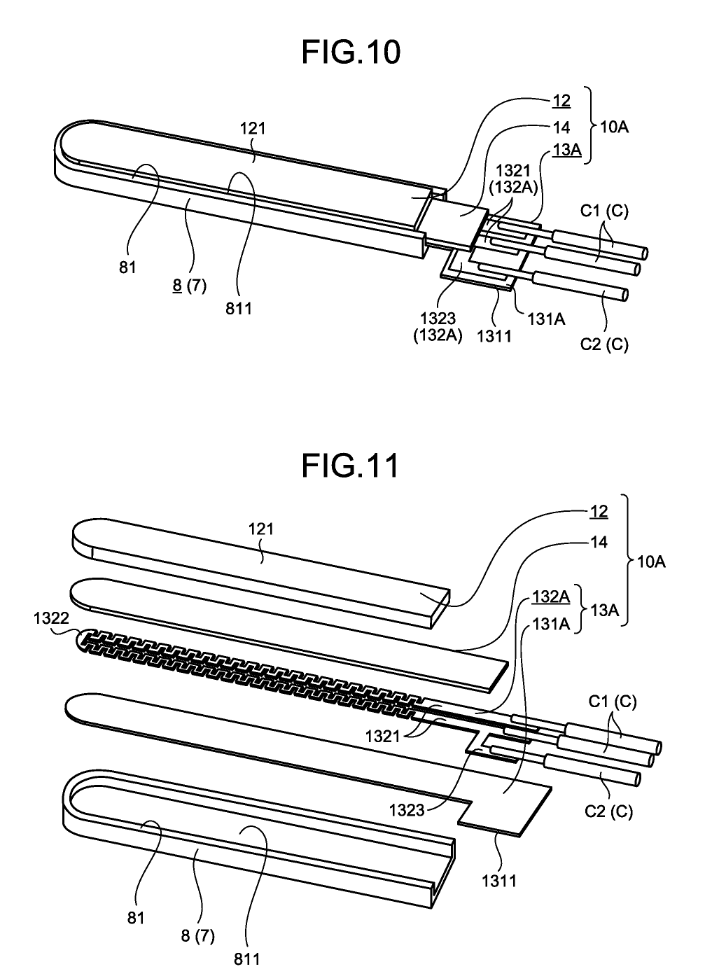

FIG. 10 and FIG. 11 are views illustrating a modification example of the first embodiment of the disclosure. Specifically, FIG. 10 is a view corresponding to FIG. 3. FIG. 11 is a view corresponding to FIG. 4.

In the above-described first embodiment, as the term (α(t)×I(t)×I(t)×Δt) corresponding to heat generation of the connectors 1321 in Formula (1), the current value I that is supplied (energized) to the energy generation unit 10 is used, but a voltage value or an electric power value may be used instead of the current value I without limitation. In this manner, in a case where the voltage value or the electric power value is used in Formula (1) instead of the current value I, it is preferable to use an energy generation unit 10A illustrated in FIG. 10 or FIG. 11 instead of the energy generation unit 10.

As illustrated in FIG. 10 or FIG. 11, the energy generation unit 10A employs a flexible substrate 13A including an insulating substrate 131A having a shape different from that of the insulating substrate 131 and a wiring pattern 132A having a shape different from that of the wiring pattern 132 in contrast to the energy generation unit 10 (FIG. 3 and FIG. 4) described in the first embodiment.

The wiring pattern 132A includes a second connector 1323 in contrast to the wiring pattern 132. Hereinafter, for convenience of explanation, the pair of connectors 1321 is described as a first connector 1321.

The second connector 1323 is a portion that is branched at a portion on a further heater 1322 side in comparison to a connection position of the lead wire C1 in one connector 1321 between the pair of connectors 1321. In addition, a second lead wire C2 that constitutes the electric cable C is bonded (connected) to the second connector 1323. Hereinafter, for convenience of explanation, the two lead wires C1 are described as a first lead wire C1.

In contrast to the insulating substrate 131, a wide-width unit 1311 of which a width dimension is wider than that of other portions is provided at one end (right end side in FIG. 10 and FIG. 11) of the insulating substrate 131A in correspondence with formation of the second connector 1323.

In addition, a control unit 33 according to this modification example acquires at least one of a current value and a voltage value V which are energized to the energy generation unit 10A (wiring pattern 132A) by using the first lead wire C1 on one side and the second lead wire C2. In addition, in Formula (1), in the case of using an electric power value instead of the current value I, the electric power value P is calculated from the current value and the voltage value V which are acquired. In addition, a temperature estimation unit 3312 according to this modification example calculates the estimation temperature T_{conduction} by using α(t)×V(t)×V(t)×Δt or α(t)×P(t)×Δt instead of α(t)×I(t)×I(t)×Δt as a term corresponding to heat generation of the connector 1321 in Formula (1).

Furthermore, in the case of employing the energy generation unit 10A illustrated in FIG. 10 or FIG. 11, it is possible to calculate resistance of the first connector 1321 by using the first lead wire C1 on one side and the second lead wire C2. That is, when using a relationship of a resistance value and a temperature of the first connector 1321 which are calculated in advance through an experiment, the resistance of the first connector 1321 which is calculated is converted into a temperature, and the temperature can be calculated as the estimation temperature T_{conduction}. According to this, in the case of employing the energy generation unit 10A illustrated in FIG. 10 or FIG. 11, the estimation temperature T_{conduction} may be calculated from the resistance value of the first connector 1321 without using Formula (1).

In addition, a configuration in which the second lead wire C2 is directly bonded (connected) to one of the first connectors 1321 may be employed without providing the second connector 1323.

### Second Embodiment

Next, a second embodiment of the disclosure will be described.

In the following description, the same reference numeral will be given to the same configuration as in the first embodiment, and detailed description thereof will be omitted or simplified.

A thermal energy treatment device according to the second embodiment is different from the thermal energy treatment device 1 described in the first embodiment in the calculation method of the estimation temperature T_{conduction}.

Hereinafter, description will be sequentially given of a configuration of the thermal energy treatment device according to the second embodiment, and an operation of a control device that constitutes the thermal energy treatment device.

Configuration of Thermal Energy Treatment Device

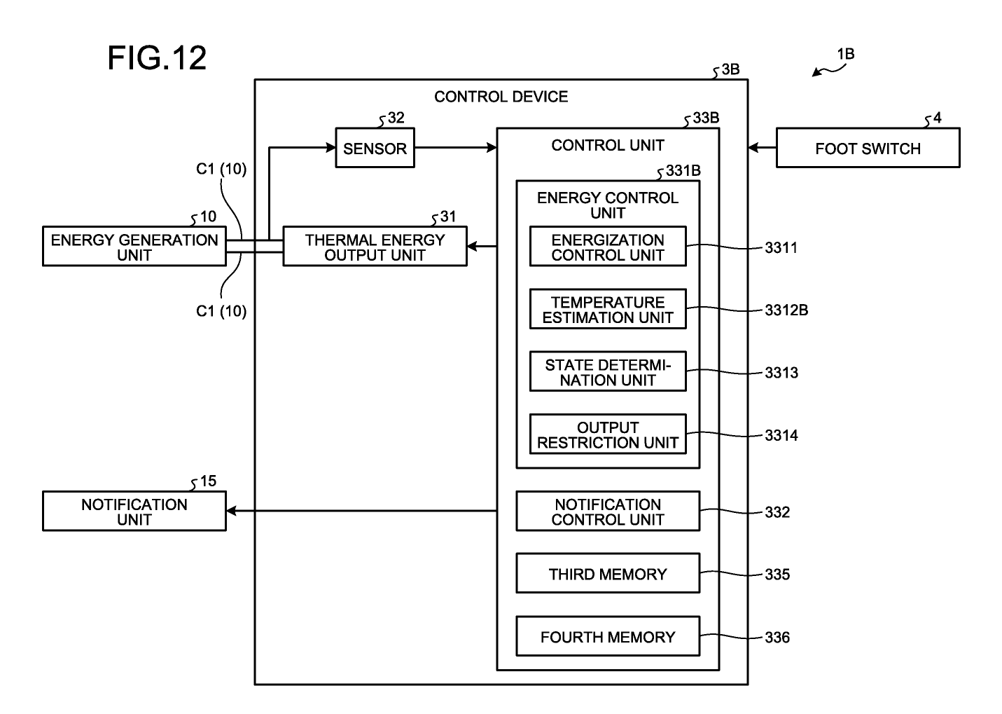

FIG. 12 is a block diagram illustrating a control device 3B that constitutes a thermal energy treatment device 1B according to the second embodiment of the disclosure.

As illustrated in FIG. 12, the thermal energy treatment device 1B employs the control device 3B (control unit 33B (energy control unit 331B)) in which the first and second memories 333 and 334 are omitted, a fourth memory 336 is added, and a temperature estimation unit 3312B is used instead of the temperature estimation unit 3312 in contrast to the thermal energy treatment device 1 (FIG. 5) described in the first embodiment.

On the basis of a current value I that is energized to the energy generation unit 10, the temperature estimation unit 3312B calculates a first heat quantity that occurs in the connector 1321 due to the energization. In addition, the temperature estimation unit 3312B calculates a second heat quantity that occurs in the connector 1321 due to heat exchange with the outside of the treatment tool 2 on the basis of an environment temperature. In addition, the temperature estimation unit 3312B calculates a third heat quantity that occurs in the connector 1321 due to heat exchange with the heater 1322 on the basis of the heater temperature. In addition, the temperature estimation unit 3312B calculates the estimation temperature T_{conduction} on the basis of the first to third heat quantities.

The first and second weighting coefficients described in the first embodiment are not stored in a third memory 335 according to the second embodiment, and a control program that is executed by the control unit 33B, and an assumed environment temperature (approximately 37° C. to 40° C. because use in a living body is assumed) on an outer side of the treatment tool 2 are stored in the third memory 335. In addition, heat capacity and a resistance value of the connector 1321, and first and second constants which are calculated in advance through an experiment are stored in the third memory 335.

The fourth memory 336 stores the estimation temperature T_{conduction} that is calculated by the temperature estimation unit 3312B. Furthermore, the fourth memory 336 stores only an estimation temperature T_{conduction} that is calculated before (immediately before) one step in the temperature estimation unit 3312B. Hereinafter, for convenience of explanation, the estimation temperature T_{conduction} that is stored in the fourth memory 336 is described as an estimation temperature T_{conduction}′ before one step.

Operation of Control Device

Next, description will be given of an operation of the control device 3B.

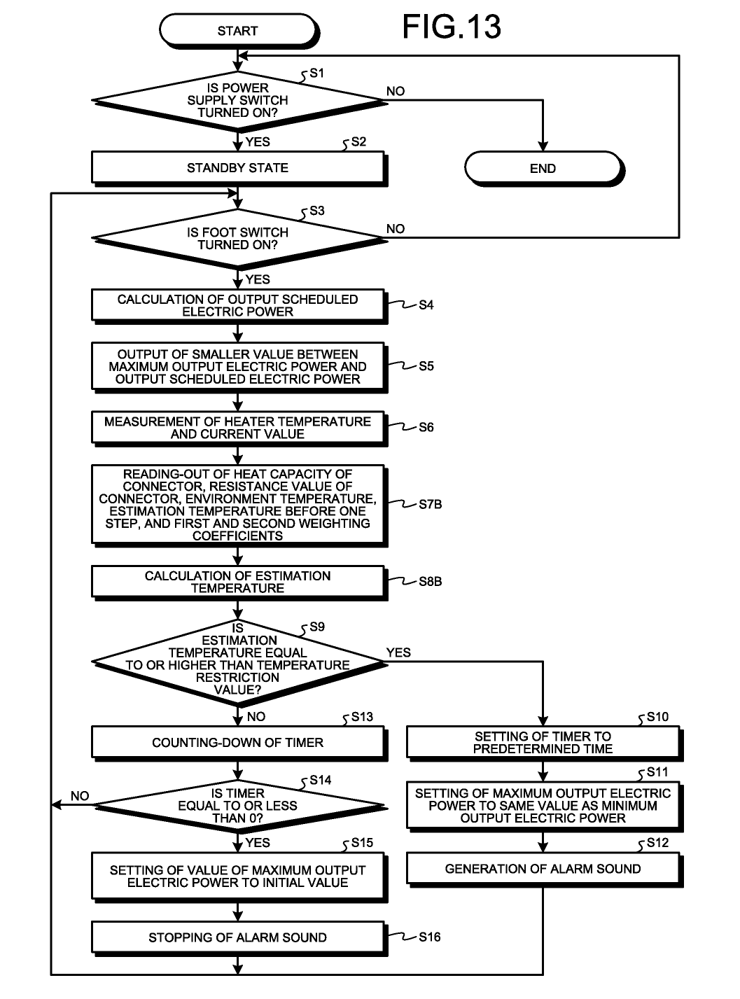

FIG. 13 is a flowchart illustrating the operation of the control device 3B.

In the operation of the control device 3B according to the second embodiment, as illustrated in FIG. 13, with regard to the operation (FIG. 6) of the control device 3 described in the first embodiment, Steps S7B and S8B are employed instead of Steps S7 and S8. Accordingly, description will be given of only Steps S7B and S8B.

In Step S7B, the temperature estimation unit 3312B reads out heat capacity C [J/K] and a resistance value R [Ω] of the connector 1321, an environment temperature [° C.], an estimation temperature T_{conduction}′ [° C.] before (immediately before) one step, a first constant θₒ [° C./(J·s)], and a second constant θ_{b} [° C./(J·s)] from the third and fourth memories 335 and 336.

In addition, in Step S8B, the temperature estimation unit 3312B calculates the estimation temperature as follows.

First, the temperature estimation unit 3312B substitutes the current value I [A] measured in Step S6 and the resistance value R [Ω] of the connector 1321 which is read out in Step S7B for the following Formula (2), and calculates a first heat quantity Qₑ [J] that is generated in the connector 1321 due to energization. Furthermore, for example, a sampling interval Δt in Formula (2) is 0.05 s (this is true of the following Formulae (3) and (4)).

Qₑ=RI²Δt (2)

Next, the temperature estimation unit 3312B substitutes the environment temperature Tₐₜₘₒₛₚₕₑᵣₑ [° C.], the first constant θₒ [° C./(J·s)], and the estimation temperature T_{conduction}′ [° C.] before (immediately before) one step which are read out in Step S7B for the following Formula (3), and calculates a second heat quantity Qₒ [J] that is generated in the connector 1321 due to heat exchange with the outside of the treatment tool 2.

$${Q}_{o} = \frac{( {T}_{atmosphere} - {T}_{conduction}^{′} )}{{θ}_{o}} Δ t \qquad ( 3 )$$

Next, the temperature estimation unit 3312B substitutes the heater temperature Tₕₑₐₜₑᵣ [° C.] that is measured in Step S6, and the second constant θ_{b} [° C./(J·s)], and the estimation temperature T_{conduction}′ [° C.] before (immediately before) one step which are read out in Step S7B for the following Formula (4), and calculates a third heat quantity Q_{b} [J] that is generated in the connector 1321 due to heat exchange with the heater 1322.

$${Q}_{o} = \frac{( {T}_{heater} - {T}_{conduction}^{′} )}{{θ}_{b}} Δ t \qquad ( 4 )$$

In addition, the temperature estimation unit 3312B adds the first to third heat quantities Qₑ, Qₒ, and Q_{b} together to calculate a total heat quantity Q [J] that is generated in the connector 1321, and substitutes the total heat quantity Q that is calculated, the heat capacity C [J/K] of the connector 1321, and the estimation temperature T_{conduction}′ [° C.] before (immediately before) one step, which are read out in Step S7B, for the following Formula (5) to calculate the estimation temperature T_{conduction} [° C.]. Next, the temperature estimation unit 3312B overwrites and saves the estimation temperature T_{conduction} that is calculated in the fourth memory 336.

$${T}_{conduction} = \frac{Q}{C} + {T}_{conduction}^{′} \qquad ( 5 )$$

Hereinafter, description will be given of a calculation example of the estimation temperature T_{conduction} in Step S8B.

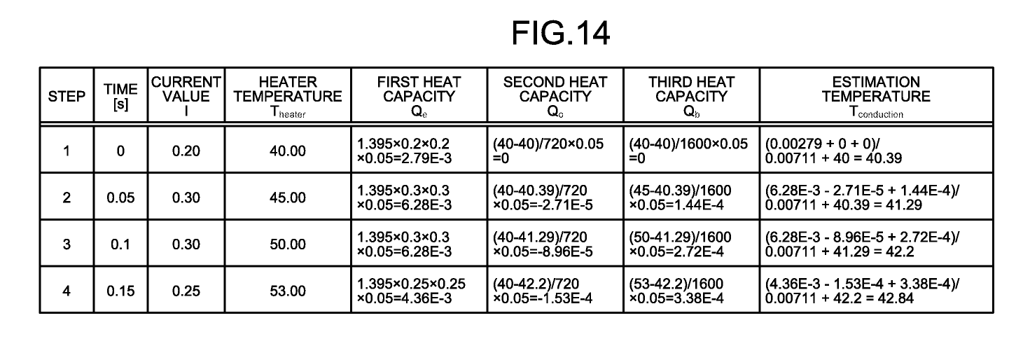

FIG. 14 is a table illustrating the calculation example in Step S8B.

In the example in FIG. 14, the environment temperature Tₐₜₘₒₛₚₕₑᵣₑ is set to 40° C., the sampling interval Δt is set to 0.05 s, the resistance value R of the connector 1321 is set to 1.395Ω, the heat capacity C of the connector 1321 is set to 0.00711 [J/K], the first constant θₒ is set to 720 [° C./(J·s)], and the second constant θ_{b} is set to 1600 [° C./(J·s)].

In addition, in the example in FIG. 14, the current value I detected by the sensor 32 in step 1 (step of calculating the estimation temperature T_{conduction} for the first time (first step when repetitively executing Steps S3 to S6, S7B, S8B, and S9 to S16)) is set to 0.20 A, and the heater temperature Tₕₑₐₜₑᵣ that is calculated by the energization control unit 3311 is set to 40° C.

In addition, in step 1, the estimation temperature T_{conduction} is calculated by using Formulae (2) to (5) in Step S8B as follows.

Specifically, the first heat quantity Qₑ becomes 1.395×0.2×0.2×0.05=2.79E-3 on the basis of Formula (2). In addition, the second heat quantity Qₒ becomes (40−40)/720×0.05=0 on the basis of Formula (3). In addition, the third heat quantity Q_{b} becomes (40−40)/1600×0.05=0 on the basis of Formula (4). In addition, the estimation temperature T_{conduction} becomes (0.00279+0+0)/0.00711+40=40.39° C. on the basis of Formula (5). Furthermore, in a case where the estimation temperature T_{conduction} is not calculated yet, and the estimation temperature T_{conduction}′ before one step is not stored in the fourth memory 336, the environment temperature Tₐₜₘₒₛₚₕₑᵣₑ is used as the estimation temperature T_{conduction}′ before one step in Formulae (3) to (5).

In addition, in the example in FIG. 14, in step 2 that is executed after step 1 by 0.05 s, the current value I detected by the sensor 32 is set to 0.30 A, and the heater temperature Tₕₑₐₜₑᵣ that is calculated by the energization control unit 3311 is set to 45° C.

In addition, in step 2, the estimation temperature T_{conduction} is calculated by using Formulae (2) to (5) in Step S8B as follows.

Specifically, the first heat quantity Qₑ becomes 1.395×0.3×0.3×0.05=6.28E-3 on the basis of Formula (2). In addition, the second heat quantity Qₒ becomes (40−40.39)/720×0.05=2.71E-5 on the basis of Formula (3). In addition, the third heat quantity Q_{b} becomes (45−40.39)/1600×0.05=1.44E-4 on the basis of Formula (4). In addition, the estimation temperature T_{conduction} becomes (6.28E-3−2.71E-5+1.44E-4)/0.00711+40.39=41.29° C. on the basis of Formula (5).

In addition, in the example in FIG. 14, in step 3 that is executed after step 2 by 0.05 s, the current value I detected by the sensor 32 is set to 0.30 A, and the heater temperature Tₕₑₐₜₑᵣ that is calculated by the energization control unit 3311 is set to 50° C.

In addition, in step 3, the estimation temperature T_{conduction} is calculated by using Formulae (2) to (5) in Step S8B as follows.

Specifically, the first heat quantity Qₑ becomes 1.395×0.3×0.3×0.05=6.28E-3 on the basis of Formula (2). In addition, the second heat quantity Qₒ becomes (40−41.29)/720×0.05=−8.96E-5 on the basis of Formula (3). In addition, the third heat quantity Q_{b} becomes (50−41.29)/1600×0.05=2.72E-4 on the basis of Formula (4). In addition, the estimation temperature T_{conduction} becomes (6.28E-3−8.96E-5+2.72E-4)/0.00711+41.29=42.2° C. on the basis of Formula (5).

In addition, in the example in FIG. 14, in step 4 that is executed after step 3 by 0.05 s, the current value I detected by the sensor 32 is set to 0.25 A, and the heater temperature Tₕₑₐₜₑᵣ that is calculated by the energization control unit 3311 is set to 53° C.

In addition, in step 4, the estimation temperature T_{conduction} is calculated by using Formulae (2) to (5) in Step S8B as follows.

Specifically, the first heat quantity Qₑ becomes 1.395×0.25×0.25×0.05=4.36E-3 on the basis of Formula (2). In addition, the second heat quantity Qₒ becomes (40−42.2)/720×0.05=−1.53E-4 on the basis of Formula (3). In addition, the third heat quantity Q_{b} becomes (53−42.2)/1600×0.05=3.38E-4 on the basis of Formula (4). In addition, the estimation temperature T_{conduction} becomes (4.36E-3−1.53E-4+3.38E-4)/0.00711+42.2=42.84° C. on the basis of Formula (5).

Hereinafter, description will be given of a calculation method of the first and second constants θₒ and θ_{b} which are used in Steps S7B and S8B.

As described above, first and second constants θₒ and θ_{b} which are used in Steps S7B and S8B are calculated in advance through an experiment, and are stored in the third memory 335.

In the experiment, first, the first constant θₒ is calculated.

Specifically, an experiment sample (U-shaped wiring pattern 132 is constituted by only the connector 1321), in which the heater 1322 is removed from the wiring pattern 132 differently from the wiring pattern 132 according to the second embodiment, is prepared. In the experiment sample, heat exchange does not occur between the heater 1322 and the connector 1321, and thus the third heat quantity Q_{b} in Formula (4) can be regarded as 0. In addition, a current I is energized to the experiment sample and a temperature (T_{conduction}) of the connector 1321 is measured (actually measured) by a temperature sensor (not illustrated) for every sampling interval Δt. In addition, the first constant θₒ is calculated from inversion from Formulae (2), (3), and (5).

Next, the second constant θ_{b} is calculated.

Specifically, after calculating the first constant θₒ, a sample having a shape of the wiring pattern 132 according to the second embodiment is used, and the current I is energized to the sample. The temperature (T_{conduction}) of the connector 1321 is measured (actually measured) by a temperature sensor (not illustrated) for every sampling interval Δt. In addition, the second constant θ_{b} is calculated through inversion from Formulae (2) to (5).

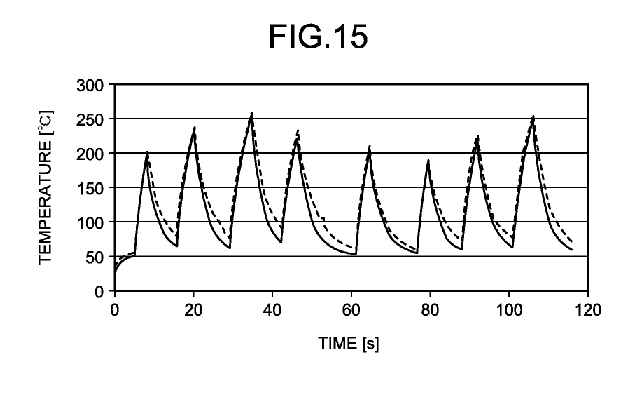

FIG. 15 is a graph illustrating an effect of the second embodiment of the disclosure. Specifically, FIG. 15 is a graph corresponding to FIG. 9, and is a graph obtained by plotting the estimation temperature T_{conduction} (indicated by a solid line in FIG. 15) that is calculated by the temperature estimation unit 3312B and the temperature (indicated by a broken line in FIG. 15) of the connector 1321 which is measured (actually measured) by a temperature sensor (not illustrated) while increasing or decreasing the current value I that is energized to the energy generation unit 10.

As in the thermal energy treatment device 1B according to the second embodiment, even in the case of calculating the estimation temperature T_{conduction} by a method different from the method in the first embodiment, as illustrated in FIG. 15, it is possible to calculate the estimation temperature T_{conduction} (solid line in FIG. 15) with high accuracy so that the estimation temperature T_{conduction} becomes approximately the same temperature as the actually measured temperature (broken line in FIG. 15), and it is possible to attain the same effect as in the first embodiment.

### Other Embodiments

Hereinbefore, embodiments for carrying out the invention have been described, but the invention is not limited to the first and second embodiments, and modification examples thereof.

In the first and second embodiments and the modification examples thereof, as the treatment unit 7, the first and second holding members 8 and 9 are opened or closed. However, there is no limitation thereto, and a configuration in which the second holding member 9 (including the heat transfer plate 92) is omitted may be employed.

In the first and second embodiments and the modification examples thereof, a configuration in which the energy generation unit 10 (10A) is provided only in the first holding member 8. However, there is no limitation thereto, and a configuration in which the energy generation unit 10 (10A) is also provided in the second holding member 9 may be employed.

In the first and second embodiments and the modification examples thereof, the thermal energy treatment devices 1 and 1B have a configuration in which thermal energy is applied to a biological tissue. However, there is no limitation thereto, and a configuration in which high frequency energy or ultrasonic energy is applied in addition to the thermal energy may be employed.

In the first and second embodiments and the modification examples thereof, the control flow is not limited to the flows illustrated in FIG. 6 and FIG. 13, and may be modified in a compatible range.

For example, in Step S12, it is not necessary for the alarm sound that is generated to be constant. As the estimation temperature T_{conduction} is high, the alarm sound may be changed to a loud sound or a high-pitched sound. In addition, in a case where the notification unit 15 is constituted by a display, for example, alarm display may be changed in correspondence with a value of the estimation temperature T_{conduction}. In addition, a configuration in which generation of the alarm sound (Step S12) and stopping of the alarm sound (Step S16) are not performed may be employed.

In addition, for example, in the output restriction in Step S11, the output value (electric power value) that is supplied (energized) to the energy generation unit 10 (10A) is restricted to the minimum output electric power (for example, 0.1 W). However, there is no limitation thereto, and supply of the output value (electric power value) to the energy generation unit 10 may be stopped.

In the first and second embodiments and the modification examples thereof, the control device 3 or 3B is provided at the outside of the treatment tool 2. However, there is no limitation thereto, and a configuration in which the control device 3 or 3B is provided at the inside of the treatment tool 2 (for example, at the inside of the handle 5) may be employed.

According to some embodiments, it is possible to attain an effect capable of avoiding an overheating state of the connector.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A thermal energy treatment device, comprising:
an insulating substrate having a longitudinal axis;
a heat generation body that is provided on one surface of the insulating substrate and includes a heater configured to generate heat due to energization and a connector that is electrically connected to the heater, the heater having a first resistance value that is a resistance value per unit length in the longitudinal axis direction, the connector having a second resistance value that is a resistance value per unit length in the longitudinal axis direction and that is smaller than the first resistance value; and
a processor comprising hardware, the processor being configured to:
energize the heater through the connector;
estimate a temperature of the connector based on at least one of a current value and a voltage value which are energized to the connector, and a temperature of the heater; and
control an output value to be energized to the heater based on the estimated temperature of the connector.

2. The thermal energy treatment device according to claim 1,
wherein the processor is configured to estimate the temperature of the connector based on at least one of the current value and the voltage value, the temperature of the heater, and an environment temperature.

3. The thermal energy treatment device according to claim 2, further comprising:
a first storage unit configured to sequentially store the temperature of the heater in association with time at which the temperature of the heater is acquired; and
a second storage unit configured to store a plurality of first weighting coefficients calculated in advance through an experiment in association with time going back to a past from a current time,
wherein the processor is configured to: calculate each difference between the environment temperature and each temperature of the heater which is sequentially stored in the first storage unit; multiply the difference by each first weighting coefficient at corresponding times; integrate a resultant value of the multiplying to obtain a first integrated value; and estimate the temperature of the connector based on at least one of the current value and the voltage value, and the obtained first integrated value.

4. The thermal energy treatment device according to claim 3, further comprising:
a third storage unit configured to sequentially store at least one of the current value and the voltage value in association with an acquisition time; and
a fourth storage unit configured to store a plurality of second weighting coefficients which are calculated in advance through an experiment in association with time going back to a past from a current time,
wherein the processor is configured to: multiply a square of each current value that is sequentially stored in the third storage unit, a square of each voltage value that is sequentially stored in the third storage unit, or each electric power value calculated from each current value that is sequentially stored in the third storage unit and each voltage value that is sequentially stored in the third storage unit by each second weighting coefficient at corresponding times; integrate a resultant value of the multiplying to obtain a second integrated value; and estimate the temperature of the connector based on the obtained second integrated value and the first integrated value.

5. The thermal energy treatment device according to claim 4,
wherein the processor is configured to estimate the temperature of the connector by adding the first integrated value, the second integrated value, and the environment temperature together.

6. The thermal energy treatment device according to claim 1,
wherein the processor is configured to: calculate a first heat quantity that is generated in the connector due to energization to the connector based on at least one of the current value and the voltage value; calculate a second heat quantity that is generated in the connector due to heat exchange with an outside of the heat generation body based on an environment temperature; calculate a third heat quantity that is generated in the connector due to heat exchange with the heater based on the temperature of the heater; and estimate the temperature of the connector based on the first heat quantity, the second heat quantity, and the third heat quantity.

7. The thermal energy treatment device according to claim 6,
wherein the processor is configured to: calculate a temperature variation of the connector by dividing a total heat quantity by heat capacity of the connector, the total heat quantity being obtained by adding the first heat quantity, the second heat quantity, and the third heat quantity; and estimate the temperature of the connector by adding the calculated temperature variation, and an immediately previously estimated temperature of the connector together.

8. The thermal energy treatment device according to claim 6,
wherein the processor is configured to:
calculate the first heat quantity by multiplying resistance of the connector, a square of the current value, and a measurement time interval;
calculate the second heat quantity by multiplying a value, which is obtained by dividing a difference between the environment temperature and an immediately previously estimated temperature of the connector by a first constant calculated in advance through an experiment, by the measurement time interval; and
calculate the third heat quantity by multiplying a value, which is obtained by dividing a difference between the temperature of the heater and an immediately previously estimated temperature of the connector by a second constant calculated in advance through an experiment, by the measurement time interval.

9. The thermal energy treatment device according to claim 7,
wherein the processor is configured to:
calculate the first heat quantity by multiplying resistance of the connector, a square of the current value, and a measurement time interval;
calculate the second heat quantity by multiplying a value, which is obtained by dividing a difference between the environment temperature and an immediately previously estimated temperature of the connector by a first constant calculated in advance through an experiment, by the measurement time interval; and
calculate the third heat quantity by multiplying a value, which is obtained by dividing a difference between the temperature of the heater and an immediately previously estimated temperature of the connector by a second constant calculated in advance through an experiment, by the measurement time interval.

10. The thermal energy treatment device according to claim 1, further comprising:
a heat transfer plate that is provided to face the one surface, the heat transfer plate being configured to transfer heat of the heat generation body to a biological tissue; and
an adhesive sheet that is provided between the one surface and the heat transfer plate, the adhesive sheet being configured to bond and fix the insulating substrate and the heat transfer plate to each other,
wherein the adhesive sheet is configured to cover an entire region of the heater, and cover a part of the connector in a state in which a part of the adhesive sheet overhangs to a connector side, and
the processor is configured to estimate a temperature of a portion exposed from the adhesive sheet in the connector.

11. The thermal energy treatment device according to claim 1, further comprising:
a first lead wire that is electrically connected to the connector; and
a second lead wire that is electrically connected to a portion on a further heater side in comparison to a connection position of the first lead wire in the connector, or to a portion that is branched at a portion on a further heater side in comparison to a connection position of the first lead wire in the connector,
wherein the processor is configured to energize the heater through the first lead wire and the connector, and
at least one of the current value and the voltage value is acquired by using the first lead wire and the second lead wire.
